(19) United States
(12) Patent Application Publication
(10) Pub. No.: US 2026/0294472 A1
(43) Pub. Date: Oct. 1, 2026

(54) SYSTEMS AND METHODS FOR ULTRASONIC GUIDED NEEDLE INSERTION

(71) Applicant: Kalysto Labs, LLC, San Diego, CA (US)

(72) Inventors: Fernando A. Sosa, San Diego, CA (US); Peter Liao, Santa Rosa, CA (US); Marc Bennett, Austin, TX (US)

(73) Assignee: Kalysto Labs, LLC, San Diego, CA (US)

(21) Appl. No.: 19/654,886

(22) Filed: Apr. 22, 2026

Related U.S. Application Data
(63) Continuation-in-part of application No. 19/467,629, filed on Feb. 2, 2026.
Application No. 19/467,629 is a continuation-in-part of application No. 19/388,383, filed on Nov. 13, 2025.
Application No. 19/388,383 is a continuation of application No. 19/184,495, filed on Apr. 21, 2025, now Pat. No. 12,491,003.
(63) Continuation-in-part of application No. 19/388,383, filed on Nov. 13, 2025.
(60) Provisional application No. 63/636,686, filed on Apr. 19, 2024.

(51) Int. Cl. A61B 17/34; A61B 8/00; A61B 8/08

(52) U.S. Cl. CPC A61B 17/3403; A61B 8/0841; A61B 8/461; A61B 2017/3413

(57) ABSTRACT

A handheld ultrasound probe for guided needle insertion into the common femoral artery comprises a linear transducer array providing longitudinal imaging and a plurality of laterally positioned transducer elements providing simultaneous cross-sectional views. The probe housing integrates both transducer configurations in a fixed spatial relationship, with the lateral elements strategically spaced to ensure needle visualization across varying tissue depths. The device generates real-time dual-plane imaging, displaying longitudinal and transverse views of the target vessel, surrounding anatomical structures, and needle trajectory on a single screen. The probe incorporates battery power capability for portable operation in emergency and field environments. The multi-plane visualization enables precise needle guidance while avoiding adjacent veins, nerves, and other critical structures during femoral artery access procedures. The compact, ergonomic design facilitates single-handed operation while maintaining optimal imaging geometry for vascular access applications.

### CROSS-REFERENCE TO RELATED APPLICATIONS

This application is a continuation-in-part application of U.S. Non-Provisional application Ser. No. 19/388,383, entitled “SYSTEMS AND METHODS FOR ULTRASONIC GUIDED NEEDLE INSERTION” and filed Nov. 13, 2025, which claims priority to U.S. Non-Provisional application Ser. No. 19/184,495, entitled “SYSTEMS AND METHODS FOR ULTRASONIC GUIDED NEEDLE INSERTION” and filed Apr. 21, 2025, which claims priority to U.S. Provisional Patent Application No. 63/636,686, entitled “SYSTEMS AND METHODS FOR ULTRASONIC GUIDED NEEDLE INSERTION” and filed on Apr. 19, 2024. The contents of the above identified applications are incorporated herein by reference in their entirety.

This application is a continuation-in-part application of U.S. Non-Provisional application Ser. No. 19/467,629, entitled “SYSTEMS AND METHODS FOR ULTRASONIC GUIDED NEEDLE INSERTION” and filed Feb. 2, 2026, which is a continuation-in-part application of U.S. Non-Provisional application Ser. No. 19/388,383, entitled “SYSTEMS AND METHODS FOR ULTRASONIC GUIDED NEEDLE INSERTION” and filed Nov. 13, 2025, which is a continuation of U.S. Non-Provisional application Ser. No. 19/184,495, entitled “SYSTEMS AND METHODS FOR ULTRASONIC GUIDED NEEDLE INSERTION” and filed Apr. 21, 2025, which claims priority to U.S. Provisional Patent Application No. 63/636,686, entitled “SYSTEMS AND METHODS FOR ULTRASONIC GUIDED NEEDLE INSERTION” and filed on Apr. 19, 2024. The contents of the above identified applications are incorporated herein by reference in their entirety.

### BACKGROUND

### Field of Art

The present disclosure is related to the field of medical ultrasound imaging devices, and more particularly to portable ultrasound-guided needle insertion systems having dual-plane imaging capabilities for real-time visualization of vascular access procedures, specifically for cannulation of blood vessels.

### Discussion of the State of the Art

Cannulation of blood vessels, such as common femoral artery (CFA), is a fundamental vascular access procedure performed across multiple medical specialties including interventional cardiology, vascular surgery, critical care medicine, and emergency medicine. The CFA serves as a primary access point for numerous diagnostic and therapeutic interventions, including cardiac catheterization, endovascular procedures, hemodynamic monitoring, and emergency resuscitation. Despite its clinical importance, CFA cannulation remains associated with significant procedural risks and technical challenges that can lead to serious patient complications.

The anatomical location of the CFA presents inherent difficulties for safe vascular access. The artery lies within the femoral triangle in close proximity to several critical structures, including the femoral vein medially, the femoral nerve laterally, and surrounding lymphatic vessels. Inadvertent puncture of these adjacent structures during CFA cannulation attempts can result in severe complications. Venous puncture may lead to arteriovenous fistula formation or compromise of arterial compression following the procedure. Femoral nerve injury can cause persistent neuropathy, chronic pain, and functional impairment. Lymphatic vessel damage may result in lymphocele formation, infection, or chronic lymphedema.

The depth of the CFA from the skin surface exhibits substantial variability across patient populations, creating additional procedural complexity. Clinical studies have demonstrated that CFA depth can range from as shallow as 5 millimeters in thin or pediatric patients to greater than 70 millimeters in obese individuals. This wide anatomical variation necessitates significant adjustments in needle insertion angle, depth estimation, and procedural technique for each patient. The inability to accurately visualize the CFA and surrounding structures throughout this depth range contributes to higher complication rates, particularly in patients at the extremes of body habitus.

Real-time ultrasound guidance has become increasingly adopted for vascular access procedures to improve safety and success rates. However, current ultrasound imaging approaches for CFA cannulation face several fundamental limitations. Traditional ultrasound transducers provide visualization in only a single imaging plane at any given time, typically either transverse (short-axis) or longitudinal (long-axis) relative to the vessel. This single-plane limitation forces clinicians to mentally reconstruct the three-dimensional relationship between the advancing needle and target vessel, a cognitively demanding task that becomes increasingly error-prone under the time pressures of emergency situations or during complex procedures.

The single-plane imaging constraint also necessitates frequent probe repositioning during the procedure to alternate between viewing planes or track needle advancement. Each probe movement risks loss of needle visualization and spatial orientation, potentially leading to unintended needle redirection and tissue trauma. Studies have shown that loss of needle tip visualization during vascular access procedures correlates with increased complication rates and procedural failure.

The technical demands of CFA cannulation often require needle insertion at relatively steep angles, typically approaching 45 degrees relative to the skin surface. At these insertion angles, conventional ultrasound transducers struggle to maintain adequate needle visualization due to fundamental acoustic physics limitations. The specular reflection from the needle shaft becomes increasingly weak as the angle between the ultrasound beam and needle increases, resulting in poor needle conspicuity precisely when visualization is most critical. Additionally, beam steering to these angles in conventional arrays often generates grating lobe artifacts that further degrade image quality.

Existing ultrasound systems capable of simultaneous multi-plane imaging are predominantly large, cart-based platforms designed for comprehensive diagnostic imaging in hospital settings. These systems require connection to wall power, occupy significant physical space, and involve substantial capital investment. Their size and infrastructure requirements make them impractical for many clinical scenarios where CFA access is required, including emergency departments with space constraints, intensive care units where bedside procedures are performed, operating rooms with limited equipment positioning options, and pre-hospital settings such as ambulances or field hospitals.

The power requirements of conventional multi-transducer ultrasound systems present additional barriers to portable implementation. Traditional beamforming architectures and multi-channel processing systems consume substantial electrical power, necessitating connection to AC mains power or large, heavy battery systems that compromise portability. This power constraint has historically prevented the development of truly portable multi-plane imaging systems suitable for point-of-care vascular access applications.

Current handheld and portable ultrasound devices designed for general imaging applications lack the specialized capabilities required for optimal CFA cannulation guidance. Generic transducer configurations are not optimized for the specific imaging requirements of femoral vascular access, including the need to visualize structures across the full range of CFA depths while maintaining needle conspicuity at steep insertion angles. The element spacing and array geometry of general-purpose transducers often result in suboptimal beam characteristics for the near-field to mid-field imaging depths where the CFA typically resides.

Phased array transducers commonly used in portable ultrasound systems exhibit inherent near-field imaging limitations that become particularly problematic when the CFA lies at shallow depths. The small aperture and element spacing of phased arrays create a natural blind spot in the superficial tissues, precisely where needle visualization is critical for shallow CFA locations. This near-field degradation can result in complete loss of needle visualization during the most critical phase of vessel puncture.

The lack of integrated, application-specific solutions for portable CFA access has significant clinical implications. Complication rates for CFA cannulation remain unacceptably high, with studies reporting major complication rates between 1-5% even with ultrasound guidance. These complications increase patient morbidity, extend hospital stays, and substantially increase healthcare costs. In emergency and resource-limited settings where portable solutions would be most valuable, the absence of appropriate technology forces clinicians to rely on landmark-based techniques with even higher complication rates.

The current state of ultrasound-guided CFA access thus presents a critical unmet clinical need for a portable, power-efficient system capable of providing simultaneous multi-plane imaging optimized for the specific anatomical and procedural requirements of femoral arterial cannulation across diverse patient populations and clinical settings.

### SUMMARY

The present invention relates to a specialized handheld ultrasound probe system designed to significantly improve the safety, accuracy, and efficiency of needle insertion procedures into the common femoral artery (CFA). The invention addresses critical limitations in current ultrasound-guided vascular access techniques by providing simultaneous, real-time visualization of both the needle trajectory and the needle tip position relative to the target vessel and surrounding anatomical structures.

The invention's unique multi-array architecture delivers procedural safety by enabling continuous visualization of the needle throughout its entire insertion path. Unlike conventional single-view ultrasound systems that require probe repositioning and can lose sight of the needle during critical moments, this invention maintains constant visual contact with both the needle shaft and tip. This dual-view capability dramatically reduces the risk of inadvertent puncture of adjacent structures, including the femoral vein, femoral nerve, and lymphatic vessels, which are common complications in CFA access procedures.

The probe's innovative combination of linear and transverse array geometry—comprising one longitudinal linear array and three strategically positioned transverse phased arrays—ensures optimal needle visualization across the full range of anatomical variations encountered in clinical practice. The array spacing guarantees that regardless of a patient's CFA depth (ranging from approximately 5 mm to 44 mm in most patients), the needle puncture point will always be visible in at least one transverse array while maintaining clinically acceptable needle insertion angles.

The invention's battery-powered design represents a significant advancement in procedural flexibility and emergency response capability. By implementing selective-activation electronics that intelligently manage power consumption while maintaining simultaneous multi-view imaging, the probe can operate independently of wall power. This portability enables its use in diverse clinical settings including emergency departments, intensive care units, ambulances, and field hospitals where immediate vascular access may be life-saving but electrical infrastructure is limited or unavailable. Additionally, conventional imaging labs, such as catheter labs where interventional cardiology procedures are conducted, often have constraints resulting in bulkier ultrasound imaging equipment being positioned at a distance from the patient being imaged which may cause difficulties when trying to control the imaging equipment while simultaneously manipulating the ultrasound probe, an issue which is addressed with the compact, portable design of the present invention.

The fixed-geometry design eliminates the need for probe rotation during procedures, a common requirement with conventional ultrasound systems that introduces multiple risks. By maintaining a stable probe position throughout the procedure, the invention reduces the likelihood of sterile field contamination, minimizes the risk of losing visualization of critical structures, and simplifies the procedural workflow. This stability is particularly valuable in emergency situations where speed and reliability are paramount.

The probe's optimized acoustic design delivers superior image quality while maintaining practical clinical usability. The longitudinal linear array, operating at 6 MHz with elements at 1.5λ pitch according to an embodiment, provides exceptional needle visualization at the standard 45° insertion angle with a generous ±10° acceptance range. This tolerance accommodates natural variations in clinician technique without compromising image quality. The transverse phased arrays, each comprising elements at 0.7λ pitch, deliver adequate beam steering capability and lateral resolution for reliable vessel visualization across the entire clinical depth range.

The incorporation of an acoustic boot pad surrounding the arrays eliminates superficial blind spots that commonly plague conventional ultrasound systems in the near field. This design ensures complete visualization from the skin surface to the deepest anatomical structures, preventing loss of needle visualization during the critical initial penetration phase.

The integrated system architecture provides significant advantages in manufacturing efficiency and long-term reliability. By optimizing the array count to exactly three transverse arrays—rather than four or more—the invention reduces component complexity, lowering production costs while maintaining full clinical functionality. The selective activation electronics not only enable battery operation but also minimize heat generation within the handheld housing, enhancing clinician comfort during extended procedures and improving electronic component longevity.

The acoustic stack implementation using 1-3 composite piezoelectric material with PZT-5H provides excellent sensitivity and bandwidth at the 6 MHz operating frequency, ensuring consistent performance across the probe's operational lifetime. The alumina-loaded silicone lens with convex geometry maintains reliable acoustic coupling while providing appropriate elevation focusing at the critical 40 mm depth where many CFA punctures occur.

The invention's calibrated depth ranges for each transverse array (Array 1: 5-10.3 mm, Array 2: 10.3-21.3 mm, Array 3: 21.3-44 mm) provide intuitive guidance for optimal probe positioning based on pre-procedure vessel assessment. This systematic approach reduces procedure time and improves first-attempt success rates. For patients with unusually deep vessels exceeding 44 mm, the calibrated linear offset strategy allows clinicians to maintain visualization by simply shifting the needle entry point along the probe surface—a straightforward adjustment that requires no additional training or equipment, however the probe may be configured to provide additional guidance regarding a desired or preferred needle entry point based on depth of an anatomical target as described below.

The simultaneous visualization of surrounding anatomical structures in the transverse views provides comprehensive safety context throughout the procedure. Clinicians can continuously monitor the positions of the femoral vein, femoral nerve, and lymphatic structures, enabling real-time adjustments to avoid these critical structures. This comprehensive awareness significantly reduces the incidence of procedure-related complications and improves patient outcomes.

In summary, the present invention represents a fundamental advancement in ultrasound-guided vascular access technology, combining innovative acoustic design, intelligent power management, and ergonomic optimization to deliver a practical, reliable, and safe solution for CFA cannulation across diverse clinical environments.

### BRIEF DESCRIPTION OF THE DRAWINGS

The accompanying drawings (if any) illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1a illustrates an exemplary embodiment of an ultrasound imaging and needle guiding system.

FIG. 1b illustrates an ultrasound device and holder of an embodiment of an ultrasound imaging and needle guiding device.

FIG. 1c illustrates an ultrasound imaging device of an embodiment of an ultrasound imaging and needle guiding device.

FIG. 1d illustrates an exemplary ultrasound imaging and needle guiding device according to an embodiment of the invention.

FIG. 1e illustrates an exemplary ultrasound imaging and needle guiding device of an embodiment of an ultrasound imaging and needle guiding device.

FIG. 1f illustrates an exemplary ultrasound imaging and needle guiding device of an embodiment of an ultrasound imaging and needle guiding device.

FIG. 2a illustrates an exemplary ultrasound probe according to an embodiment of the invention.

FIG. 2b illustrates an exemplary transducer array according to an embodiment of the invention.

FIG. 2c illustrates an exemplary ultrasound probe according to an embodiment of the invention.

FIG. 2d illustrates an exemplary embodiment of a needle offset region according to an embodiment of the invention.

FIG. 3 illustrates an exemplary ultrasound probe according to an embodiment of the invention.

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of an exemplary architecture that supports an embodiment of the inventive disclosure.

FIG. 7 illustrates components of a computing device that supports an embodiment of the inventive disclosure.

### DETAILED DESCRIPTION OF EMBODIMENTS

The invention is described by reference to various elements herein. It should be noted, however, that although the various elements of the inventive apparatus are described separately below, the elements need not necessarily be separate. The various embodiments may be interconnected and may be cut out of a singular block or mold. The variety of different ways of forming an inventive apparatus, in accordance with the disclosure herein, may be varied without departing from the scope of the invention.

Generally, one or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices and parts that are connected to each other need not be in continuous connection with each other, unless expressly specified otherwise. In addition, devices and parts that are connected with each other may be connected directly or indirectly through one or more connection means or intermediaries.

A description of an aspect with several components in connection with each other does not imply that all such components are required. To the contrary, a variety of optional components may be described to illustrate a wide variety of possible embodiments and in order to more fully illustrate one or more embodiments. Similarly, although process steps, method steps, or the like may be described in a sequential order, such processes and methods may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, or method is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Alternate implementations are included within the scope of various embodiments in which, for example, functions may be executed out of order from that shown or discussed, including substantially concurrently or in reverse order, depending on the functionality involved, as would be understood by those having ordinary skill in the art.

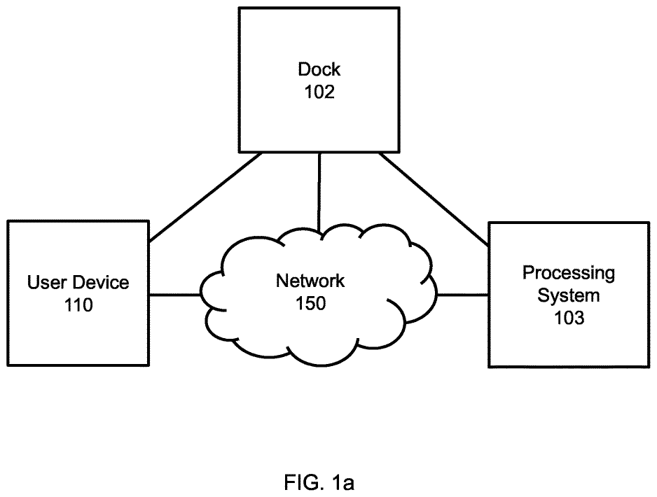

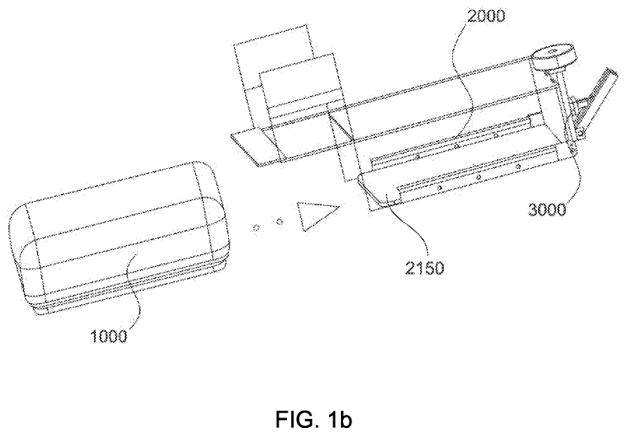

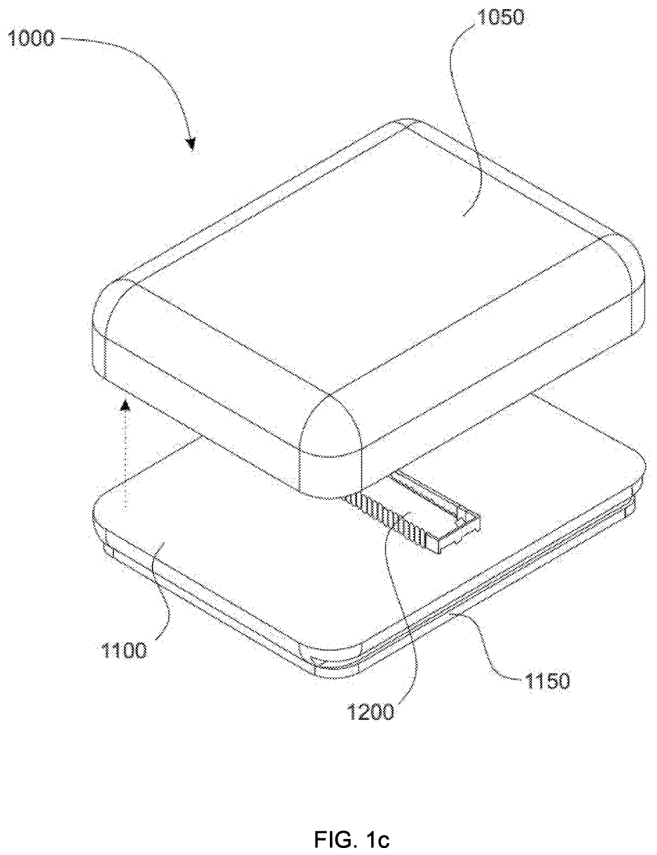

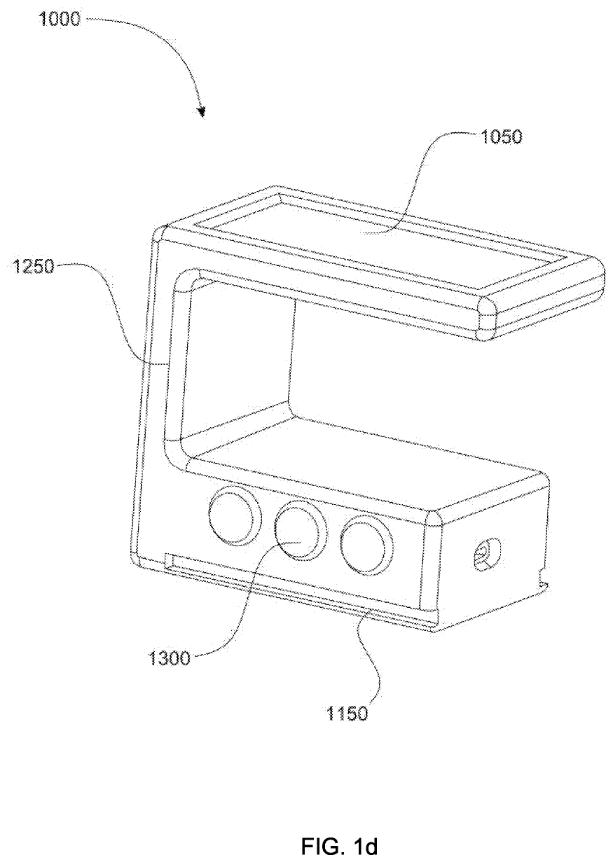

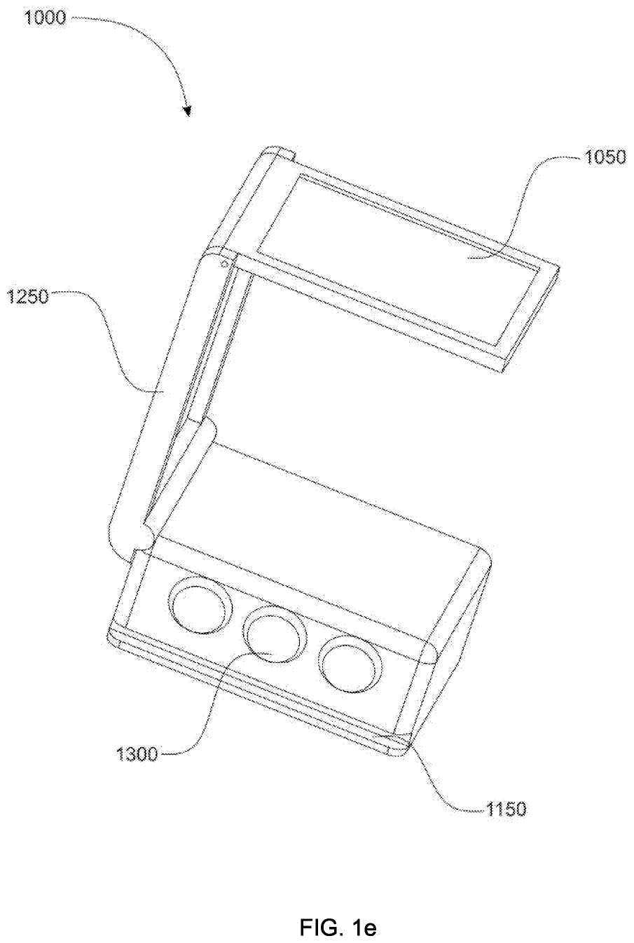

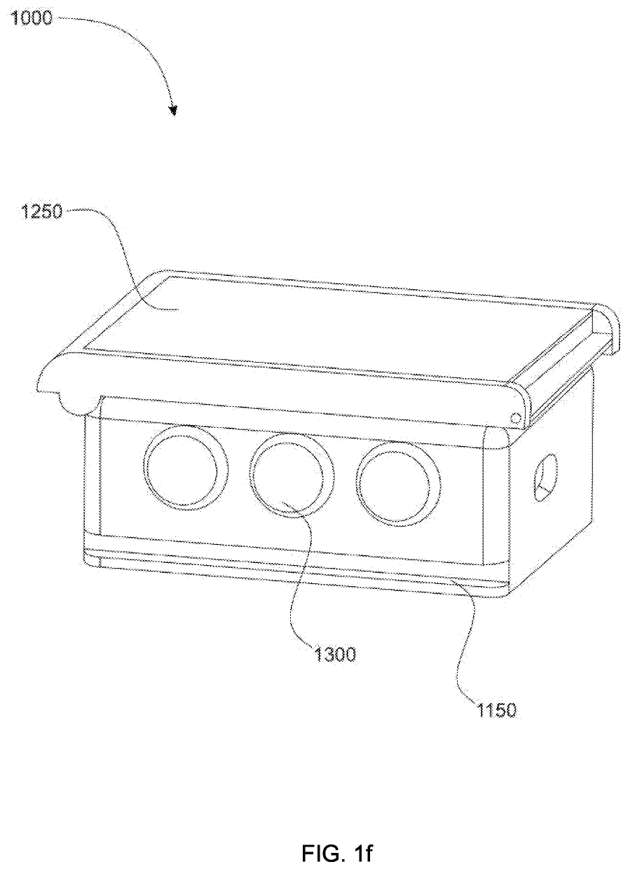

FIG. 1a illustrates an exemplary embodiment of a system for ultrasound guided needle insertion according to one embodiment. The system includes a user device 110, dock 102, processing system 103, and a network 150 over which the various systems communicate and interact. The various components described herein are exemplary and for illustration purposes only and any combination or subcombination of the various components may be used as would be apparent to one of ordinary skill in the art. The system may be reorganized or consolidated, as understood by a person of ordinary skill in the art, to perform the same tasks on one or more other servers or computing devices without departing from the scope of the invention.

User device 110 may comprise an ultrasound device (e.g. ultrasonic needle guidance system 1500, handheld ultrasound unit 1000, etc.). The ultrasound device may comprise an ultrasound probe. The ultrasound device may be a handheld device for acquiring images of a patient and/or providing feedback to a user. The user device 110 may comprise a boot operable to engage with ultrasound probe. The boot may provide ultrasonic coupling between the probe and the patient. The boot may comprise a needle guide. The needle guide may comprise an adjustment mechanism allowing adjustment of the angle of the needle guide. The user device 110 is generally operable to obtain images of patient anatomy, process the images and/or transmit the images to an external component for processing, and display feedback to the user. The feedback may comprise at least one of images of the anatomy, a recommended needle guide angle, and information indicating to the user where to move or position the user device during a procedure. Additional details of the user device 110 are provided below and throughout the description.

User device(s) 110 include, generally, a computer or computing device including functionality for communicating (e.g., remotely) over a network 150. Data may be collected from user devices 110, and data requests may be initiated from each user device 110. User device(s) 110 may be a server, a desktop computer, a laptop computer, personal digital assistant (PDA), an in-or out-of-car navigation system, a smart phone or other cellular or mobile phone, or mobile gaming device, among other suitable computing devices. User devices 110 may execute one or more applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 110 may be an electronic device including hardware, software, or embedded logic components or a combination of two or more such components and capable of carrying out the appropriate functions implemented or supported by the user device 110. For example and without limitation, a user device 110 may be a desktop computer system, a notebook computer system, a netbook computer system, a handheld electronic device, or a mobile telephone. The present disclosure contemplates any user device 110. A user device 110 may enable a network user at the user device 110 to access network 150. A user device 110 may enable its user to communicate with other users at other user devices 110.

A user device 110 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 110 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 110 one or more Hyper Text Markup Language (HTML) files responsive to the HTTP request. The user device 110 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 110 may also include an application that is loaded onto the user device 110. The application obtains data from the network 150 and displays it to the user within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

Network cloud 150 generally represents a network or collection of networks (such as the Internet or a corporate intranet, or a combination of both) over which the various components illustrated in FIG. 1a (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 421 or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client/user devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiment, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1a, but would be readily apparent to a person of ordinary skill in the art. Other databases and systems may be added or subtracted, as would be readily understood by a person of ordinary skill in the art, without departing from the scope of the invention.

Dock 102 is operable to interface with user device 110 and provide a data communication interface and/or a power supply interface. Dock 102 may transmit data between the user device 110 and processing system 103 via direct communication and/or via network 150. In one aspect, dock 102 may comprise processing hardware and/or software suitable for processing obtained images without the need for communication with a separate processing system 103. For example, the dock may be operable to execute the processing operations of the processing system as described below.

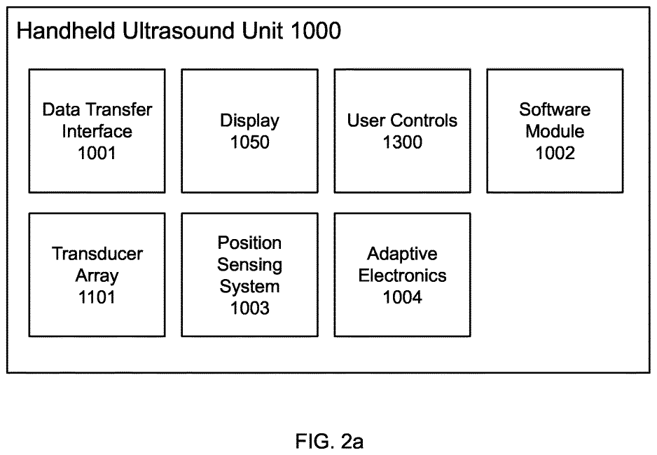

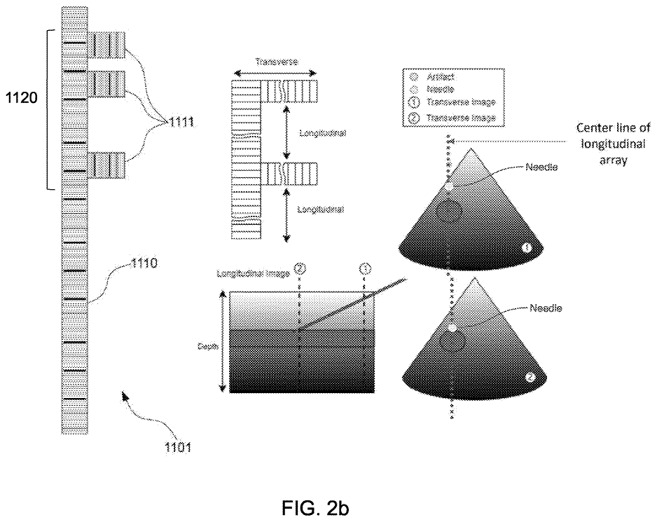

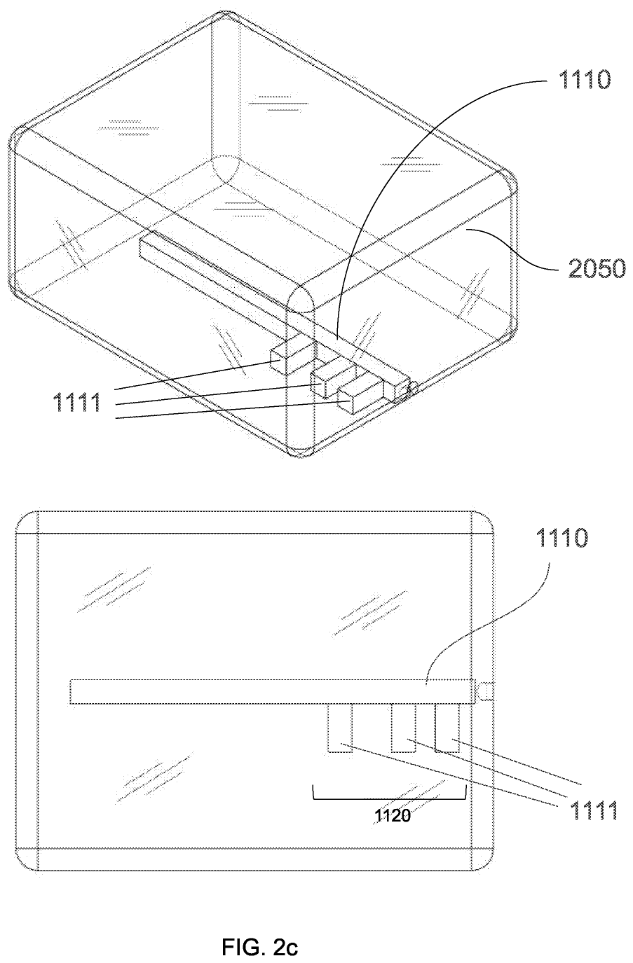

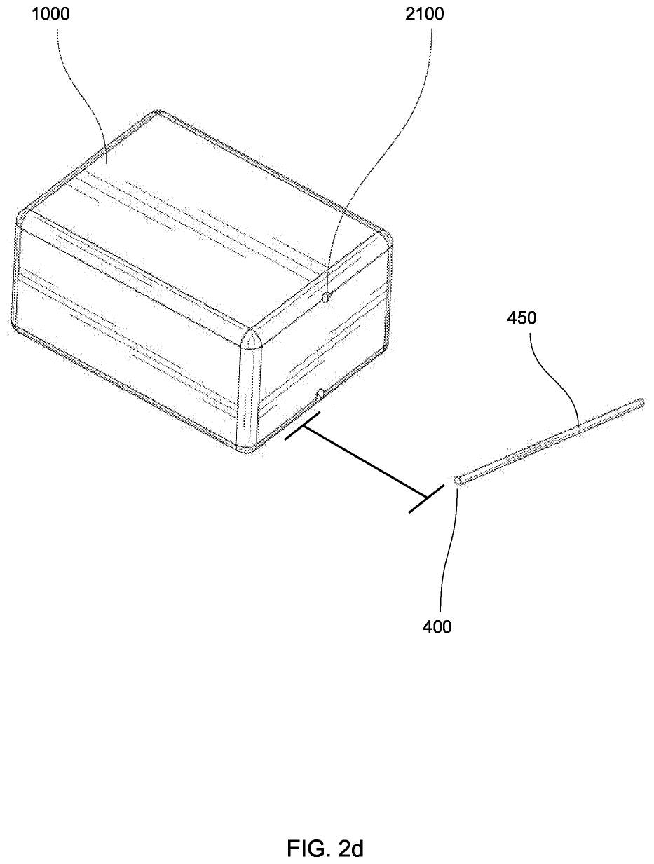

Processing system 103 is operable to process obtained images and determine feedback to be provided to a user via user device 110. Although depicted as a separate component in FIG. 1a, processing system 103 may be incorporated into dock 102 as discussed above and/or incorporated into user device 110 (e.g. as software module 1002 as depicted in FIG. 2a). Processing system 103 may analyze obtained images using artificial intelligence (AI) and/or machine learning (ML) in order to identify anatomy and/or determine at least one of a target puncture site of a blood vessel and a target needle guide angle.

FIG. 1b illustrates an example device capable of executing the software described in the invention. The device may comprise a handheld ultrasound unit 1000, a boot 2000, and a needle guide 3000.

The handheld ultrasound unit 1000 may be used to gather images and make recommendations related to needle insertion for a medical procedure. The handheld ultrasound unit 1000 may be an ultrasound probe. In an embodiment of the invention, the medical procedure is PCI, however the device may be used with other medical procedures known in the art that require proper needle insertion. Alternatively, the handheld ultrasound unit 1000 may be used to provide imaging identification of anatomical structures without the inclusion of a needle guide. The handheld ultrasound unit 1000 may connect to a docking station through either wireless or usb connections for the purpose of exchanging of patient ultrasound imagery and AI data, for battery recharge, downloading of data, and Firmware updates.

The boot 2000 may be used to surround the ultrasound unit for use in a sterile environment. The boot 2000 may be made of plastics (e.g. clear plastics) and be disposable. The boot 2000 may comprise a waterproof, disposable sterile covering that completely encloses handheld ultrasound unit 1000 during a Percutaneous Cardiac Intervention (PCI) procedure or other related medical procedures. A user may apply jelly to the outside of the boot 2000 to improve image quality. The boot 2000 may be collapsed where the needle guide 3000 is folded down, for more compact storage and when in use, the needle guide 3000 may be upright and perpendicular to the boot.

The needle guide 3000 may provide the proper angle for needle insertion for a user. The needle guide may be adjustable and removable from the holder if the user prefers manual needle insertion. The needle guide 3000 may comprise plastics appropriate for a sterile location and be disposable after use.

FIG. 1c illustrates an embodiment of the handheld ultrasound unit 1000. The handheld ultrasound unit 1000 may comprise a display unit 1050, an ultrasound base unit 1100, a slot 1150, and a data connection 1200.

The display unit 1050 may be used to project an image gathered by the ultrasound base unit 1100. The display unit 1050 may comprise a housing, a liquid crystal display, LCD, screen and user interface components 1300. The display unit 1050 allows the user to maintain a continuous line of sight between the imaging output and the insertion site, improving hand-eye coordination and procedural accuracy. The display 1050 may receive processed image data from an onboard or external computing module and may present anatomical structures, needle guidance overlays, and system feedback through a high-resolution interface. In one embodiment, the display 1050 is fixed in position relative to the probe; however, alternative configurations may include a telescoping display 1050 that can be extended or retracted to improve visibility, or a hinged display 1050 that allows angular adjustment to reduce glare or accommodate different viewing angles. The display unit 1050 may comprise a data exchange interface comprising at least one of a physical connection and wireless connection.

The ultrasound base unit 1100 may be used to gather ultrasound data to be communicated to the display unit 1050. The ultrasound base unit 1100 may comprise a housing and an ultrasound transducer array. In an embodiment of the invention the ultrasound array may be “T” or “F” shaped, but various array shapes may be used based on the image type needed for the medical procedure. As imaging needs change, different modules may be plugged in for best ultrasound images.

The slot 1150 may be used to attach the ultrasound unit 1000 to the boot 2000. The set of slots 1150 or channels configured to engage with corresponding rails or projections on the sterile boot 2000, thereby securing the probe 1000 in a fixed and repeatable orientation relative to the boot 2000 during use. These slots 1150 may guide the insertion of the probe 1000 into the boot 2000 and ensure proper alignment of the transducer with the acoustic window, minimizing the risk of air gaps and misalignment that could degrade image quality. The mechanical interface between the slots and rails also facilitates rapid attachment and detachment, allowing for efficient setup and disposal in sterile environments. Other joining mechanisms known in the art may be used in place of the rails, such as locking fasteners.

An optional data connection 1200 may be used to communicate between the display unit 1050 and ultrasound base unit 1100. Additionally the data connection may provide a locking fit between the display unit 1050 and ultrasound base unit 1100. to the bottom of the main handheld unit to transmit ultrasound data and control the elements of the ultrasound array. The data connection 1200 may comprise a multi pin connector interface or other known connections in the art. Alternatively, wireless communication interfaces may be used.

FIG. 1d illustrates an example of the handheld ultrasound unit 1000. The handheld ultrasound unit 1000 may comprise the same components found in FIG. 1c, and additionally a rigid arm 1250 and a set of user controls 1300. The ultrasound unit 1000 may be arranged in a C shape to allow for a user to place their hand underneath the display 1050 to enable a user to manipulate the controls while still being able to view the display.

The rigid arm 1250 may comprise an integrated section of the ultrasound unit 1000 housing. The rigid arm 1250 maintains a fixed spatial relationship between the imaging plane and controls 1300 and the display 1050, enabling a user to intuitively interpret image data while manipulating the probe. By positioning the display 1050 directly in the user's line of sight and ensuring alignment with the transducer orientation, the rigid arm 1250 enhances ergonomics and visual continuity during procedures. In alternative embodiments, the rigid arm 1250 may be replaced or supplemented by a telescoping section, allowing vertical adjustment of the display height to accommodate different user preferences or clinical environments. Similarly, a hinged or pivoting section may be incorporated to allow angular adjustment or folding of the display for improved portability or storage.

User controls 1300 may comprise a user interface for allowing the user to provide input related to control of the system. User controls 1300 may comprise one or more physical controls including, but not limited to, button, knob, toggle, dial, switch, slider, and/or touch sensor. In one aspect, user controls may be embodied in display 1050 (e.g. via touchscreen controls displayed on display 1050). User controls 1300 may serve to provide various functions such as, but not limited to, switching between displayed views (e.g. between longitudinal and transverse), receiving input from a user indicating a target to be reached with a needle, storing/saving images, and the like.

FIG. 1e illustrates an example of the handheld ultrasound unit 1000. The handheld ultrasound unit 1000 may incorporate the previously described components, but arranged with a hinged display 1050 and rigid arm 1250.

FIG. 1f illustrates an example of the handheld ultrasound unit 1000. The handheld ultrasound unit 1000 is pictured in the folded up form of FIG. 1e. The display 1050 is contained within the rigid arm 1250 and protected for ease of transportation.

FIG. 2a illustrates an exemplary handheld ultrasound unit 1000 according to an embodiment of the invention. The handheld ultrasound unit 1000 (which may also be referred as a probe, ultrasound probe, etc.) comprises data transfer interface 1001, display 1050, user controls 1300, software module 1002, transducer array 1101, and position sensing system 1003. Other components, modules or the like may be included without departing from the scope of the invention.

Data transfer interface 1001 is operable to transmit data for further processing and/or receive input related to remotely processed data. Data transfer interface 1001 may transmit data for remote processing via at least one communication means (e.g. via a dock (or docking station), wired or wireless connection, etc.). Depending on the complexity of the analysis to be performed, scenarios may arise where the data (e.g. ultrasound images and/or user input) are offloaded to a remote processing system which analyzes the data and returns feedback to the handheld ultrasound unit 1000. The data transfer interface 1001 facilitates this data transmission/reception. Data transfer interface 1001 is operable to transmit data for storage at a location remote from the handheld ultrasound unit 1000.

Display 1050 is operable to display information associated with data obtained and/or generated in association with use of the ultrasonic needle guidance system 1500. Display 1050 may display ultrasound images generated by the handheld ultrasound unit 1000. Display may provide longitudinal images and/or transverse images of the underlying anatomy. Display may be configured to switch between displaying a longitudinal image and a transverse image (perpendicular to the longitudinal image). Display may be configured to simultaneously display at least one longitudinal image with at least one transverse image. Display may display a longitudinal image simultaneously with a plurality of transverse images wherein each transverse image is associated with a different imaging plane along the length of the handheld ultrasound unit. Display may be configured to display a longitudinal image while simultaneously displaying and switching between different transverse images. Displaying a plurality of transverse images (either sequentially or simultaneously) provides the benefit of being able to visualize a needle at multiple points along its insertion trajectory allowing for a better understanding of the actual needle path. Display may be configured to provide feedback to a user regarding at least one of positioning of the handheld ultrasound unit, indicating what images are needed for further processing, needle angle (determined from the guide assembly and/or image processing).

User controls 1300 may comprise a user interface for allowing the user to provide input related to control of the system. User controls 1300 may comprise one or more physical controls including, but not limited to, button, knob, toggle, dial, switch, slider, and/or touch sensor. In one aspect, user controls may be embodied in display 1050 (e.g. via touchscreen controls displayed on display 1050). User controls 1300 may serve to provide various functions such as, but not limited to, switching between displayed views (e.g. between longitudinal and transverse), receiving input from a user indicating a target to be reached with a needle, storing/saving images, and the like. The user controls or interface features tactile controls specifically positioned to allow for single-handed operation. Dedicated buttons provide immediate access to frequently utilized functions, such as image freeze, gain control, and depth adjustment. Haptic feedback is utilized to confirm mode changes and button presses, eliminating the need for the clinician to divert their visual attention away from the display.

Software module 1002 is operable to control one or more functions related to an ultrasonic needle guidance process. The software module is generally configured to at least one of: track a current needle location and/or trajectory based on input received from the needle guide assembly and/or from image processing, identify a recommended needle angle and/or trajectory, compare the current needle location and/or trajectory with the recommended needle angle/trajectory, and/or notify a user of the current needle angle/trajectory status. The software module may comprise signal processing circuitry for processing signals received from the position sensing system 1003 for determining the location of the handheld ultrasound unit 1000 (e.g. the location relative to a reference location as discussed below). The software module may comprise signal processing circuitry for processing signals received from the transducer array to generate ultrasound images. The software module 1002 may analyze ultrasound images to determine characteristics of the anatomy, needle advancement, and/or handheld ultrasound unit positioning (e.g. location and/or orientation). Software module 1002 may employ needle enhancement techniques to identify and accentuate the current location and/or projected path of the needle. The software module 1002 may process input from a needle guide assembly to determine characteristics of needle advancement. Software module 1002 may receive input from at least one of position sensing system 1003, transducer array 1101 (or from a signal processing module configured to process obtained transducer signals), and/or user controls 1300 and process the input and/or adjust display 1050 accordingly, in real-time.

The software module 1002 may comprise a processor and/or memory for processing received input and generating corresponding output. The software module 1002 may process input to identify an inserted needle and/or projected needle trajectory in a plurality of imaging planes (e.g. longitudinal and transverse) thereby allowing a user to simultaneously view progress of the needle guidance procedure from different perspectives to ensure the needle is approaching the target in a desirable manner. The software module 1002 may generate and provide at least one notification to be displayed to a user (e.g. on display 1050) as a result of the processing performed. For example, software module 1002 may notify a user of a change in expected needle trajectory status, such as a deviation from the recommended trajectory that exceeds a threshold or when an expected needle trajectory indicates the expected path will result in missing a target or indicates the expected path will intersect an anatomical structure to be avoided (e.g. a sensitive anatomical structure that could lead to patient complications if pierced by a needle).

The software module 1002 may perform image processing (locally or may be assisted by a remote processing system) to identify and accentuate needle location. Software module 1002 may generate an image depicting at least one of needle enhancement or projected needle path. Software module 1002 may employ techniques such as shading, highlighting, overlay(s), pixel blending, etc. to generate images emphasizing the needle location, target, projected needle path, and/or anatomical structures.

The software module 1002 is operable to determine appropriate processing to be performed based on the presence or absence of a needle guide assembly. The software module 1002 may determine an operational paradigm to be used based on the presence (or absence) of a needle guide assembly as determined using the position sensing system 1003 such that the software module is operable to track a needle with or without input from a physical needle guide. In one aspect, software module 1002 adjusts the operational paradigm based on user input (e.g. via user controls 1300). When a needle guide assembly is not present (e.g. as indicated by position, software module 1002 is configured to use image processing to determine and/or display needle angle and/or trajectory information. When a needle guide assembly is present, software module 1002 is configured to use input from needle guide (in particular the angle information) to determine and/or display needle angle and/or trajectory information. Note that the presence of a needle guide assembly does not require relying solely on the needle guide assembly, but instead may use both needle guide information and image processing techniques to determine and/or display needle angle and/or trajectory information. The needle guide assembly may be configured to constrain a needle angle to a certain range depending on the procedure and/or transducer being used. For example, for different procedures, depth of a target to be reached by a needle may vary thus affecting the range of angles which can be used to reach the target while maintaining sufficient capabilities to image the needle. As another example, different procedures or different target depths may involve using a different transducer array (e.g. using a modular transducer array) which is configured for a different depth which also influences the range of acceptable angles over which the needle can be viewed. In other words, steeper needle insertion angles result in less sound wave reflection by the needle (as compared to shallower, more perpendicular orientations) thereby making it more difficult to view the needle. Therefore, the needle guide assembly may constrain (e.g. with control assistance from software module 1002) the needle guide assembly to a range of needle angles suitable for imaging by the transducer array.

The adaptive electronics 1004 are housed within the probe body and are electrically coupled to both the longitudinal linear transducer array and the transverse phased array transducers. A multichannel beamforming system provides independent receive and transmit processing for each of the transducer arrays. The beamformer for the longitudinal array supports 256 channels, allowing for either multiplexed mode operations or full synthetic aperture imaging. The beamformers for the three phased arrays each support 48 channels and feature programmable delays for dynamic focusing and beam steering. The number of channels is merely exemplary based on the number of elements in each transducer array and can be adapted to have more or less as would be apparent to one of ordinary skill in the art.

Digital signal processing circuitry enables simultaneous image formation, scan conversion, and envelope detection across all four imaging planes. A central processing unit coordinates all subsystems and executes algorithms that automatically optimize imaging parameters based on depth measurements and detected tissue characteristics. Real-time image processing capabilities include automated measurement tools specific to vascular access procedures, edge enhancement, and speckle reduction. The software module utilizes needle enhancement algorithms based on image processing to increase the visibility of the needle against surrounding tissues. The adaptive electronics adjust the operation of the transducer arrays based on the inserted needle progression or the target depth. The system can dynamically select which of the transverse phased array transducers is active in response to the detected depth of the target blood vessel. To enable battery-powered handheld operation, the adaptive electronics reduce instantaneous power consumption and channel count by operating the longitudinal array with a subset of active elements and limiting the number of active transverse arrays and elements during beam steering. Intelligent power management further minimizes power usage by selectively activating only the arrays currently in use and employing aggressive power-down modes during periods of inactivity. The processor also executes safety monitoring functions, such as performing acoustic output calculations to ensure the device complies with diagnostic ultrasound regulatory limits. The adaptive electronics may operate in coordination with the software module 1002 in coordinating control of the transducer arrays based on information received and processed by the software module and/or output generated by the software module.

Transducer array 1101 generally comprises a plurality of ultrasound elements arranged to image two different planes without changing ultrasound probe positioning. The transducer array may comprise a first array 1110 for longitudinal imaging and a second array(s) 1111 for transverse imaging. The longitudinal array 1110 may comprise a linear array. The transverse arrays 1111 may comprise phased arrays. The linear array is generally designed for capturing longitudinal images along a longitudinal axis of a target (e.g. a blood vessel) when the handheld ultrasound unit is properly positioned relative to the target. The phased arrays are generally designed for capturing transverse images perpendicular to the image planes scanned by the linear, longitudinal array. The transducer array 1101 is configured to scan orthogonal dimensions simultaneously and without the need to reposition the ultrasound probe thereby allowing for real-time generation of both longitudinal and transverse images for real time anatomy and/or needle tracking

The transducer array may be modular such that different arrays having different designs and element arrangements may be used as needed for adapting the ultrasonic needle guidance system for different procedures and/or to account for different characteristics of users undergoing a procedure (e.g. accounting for changes in target or imaging depth which may vary from patient to patient). In this way, modularity of the transducer array allows for the selection of optimal arrays for a given procedure. Various beamsteering or beamforming techniques may be used to enable imaging over the desired field of view. The transducer array 1101 may be configured to perform three dimensional (3D) imaging. The transducer array may be inside or otherwise coupled to the ultrasound probe housing. Additional details of an exemplary transducer array 1101 are depicted and described in association with FIG. 2b-2c below.

Position sensing system 1003 is generally operable to determine at least one of the presence of a needle guide assembly and the angle of a corresponding needle guide rail when the needle guide assembly is present (i.e. positioned in close proximity to the handheld ultrasound unit 1000). Position sensing system 1003 may comprise at least one magnet sensor operable to detect the presence of at least one magnet. Position sensing system 1003 may comprise a first magnet sensor for detecting the presence of a first magnet in the needle guide assembly. The first magnet sensor may measure the magnetic field associated with the first magnet. When the magnetic field sensed by the magnet sensor meets certain criteria (e.g. a detected magnetic field strength exceeds a threshold value), the position sensing system 1003 determines that a needle guide assembly is present. Position sensing system 1003 may comprise a second magnet sensor in the form of a magnetic position sensing array for detecting a location of a second magnet in the needle guide assembly. The second magnet sensor may measure the magnetic field generated by the second magnet and use this information to determine where the second magnet is located along the length of the second magnet sensor (e.g. position sensing array). Position sensing system 1003 may determine the position of the handheld ultrasound unit 1000 relative to a reference position (e.g. a starting position or previously stored/recorded reference position). This relative position may be determined from user input and/or from image processing techniques configured to recognize major anatomical landmarks and determine an approximate location based on the location of the anatomical landmarks. The position awareness may employ accelerometers, gyroscopes, other position sensing devices/components and/or other motion detection devices/components to derive the relative positioning of the ultrasound unit.

FIG. 2b illustrates an exemplary transducer array and corresponding images according to an embodiment of the invention. The transducer array comprises a longitudinal linear array 1110 and a plurality of transverse phased arrays 1111. The transverse phased arrays 1111 are positioned at different locations along the length of the longitudinal array 1110. The array elements of the phased arrays are aligned perpendicular to the array elements of the longitudinal array. It is noted that while three phased arrays are shown, more or less could be used without departing from the scope of the invention as would be apparent to one of ordinary skill in the art.

Spacing of the phased arrays may be based on a combination of the depth of the target and a range of expected needle angles to be used for reaching the target. Spacing of the phased arrays may be based on a range of needle angles to which the needle should be constrained (e.g. needle angles which allow for ultrasonic detection of the needle). The spacing of the phased arrays may be configured so that one of the plurality of phased arrays can be aligned directly over the needle target such that the resulting image depicts the needle in close proximity to the target after the needle has been advanced towards the target. This configuration of array elements (sometimes referred to herein as an “F” configuration) provides various benefits over other configurations, such as a “T” or “+” based configuration of elements where phased array elements are provided on both sides of a longitudinal array. This F configuration allows for reduced cost, fewer number of transducer elements, and reduced space requirements over other configurations. Furthermore, this configuration reduces and/or eliminates the problem and complexities associated with blending of images when phased arrays are on either side of the longitudinal array. Moreover, the series of transverse image planes (three in this example) provide confidence that the device is aligned with the target and provides multiple checkpoints to confirm needle placement in the transverse plane during insertion. These transverse image planes provide context for structures around the target, adding to the safety provided by the device. For example, in the case of the target being a femoral artery, the transverse image planes may allow for identification and avoidance of nearby anatomical structures such as femoral vein, femoral nerve, lymphatics, etc. To ensure visualization of puncturing of the target with the needle, this configuration allows for adjustment of the needle entry angle based on the target puncture depth such that the puncture depth aligns with one of the transverse image planes.

A challenge with the longitudinal array is imaging the entire width of the needle trajectory while minimizing the total number of elements required. To address this, the transducer elements may be configured with pitch suitable for identifying and/or enhancing needle appearance. For example, a pitch of 1.5λ may be used for the longitudinal array as a good trade-off in reducing grating lobe levels and total element count while maintaining suitable imaging at desired depths. For the phased array(s), it is desirable to have a larger aperture, however, grating lobe artifacts must also be considered. For the phased array(s), a pitch of 0.7λ may be used as a good compromise between steerability and resolution. These are exemplary pitch values and other pitch values among other transducer configurations may be used without departing from the scope of the invention as would be apparent to one of ordinary skill in the art. The transverse array(s) may be configured to image the transverse cross section of the target (e.g. a femoral artery) as well as the needle tip at a fixed depth thereby enhancing the ability to image and view the needle in close proximity to the target to improve the likelihood that the target is punctured as desired.

It is noted that although depicted in FIG. 2b with generated images having the center line of the linear array offset relative to the center line of the transverse images, the system can be reconfigured or employ signal processing techniques to change the alignment and account for the offset as would be apparent to one of ordinary skill in the art. In addition, a different number of arrays and/or spacing of arrays could be used without departing from the scope of the invention as would be apparent to one of ordinary skill in the art.

FIG. 2c illustrates an exemplary embodiment of the transducer array configuration within a probe housing. The elements individually or in combination provide the benefits described above. Although described herein with reference to use of the system for CFA procedures, the system may be used for and/or adapted to different vascular access procedures as would be apparent to one of ordinary skill in the art.

At a high level, the present invention is for an ultrasound probe system comprising multiple integrated components working in concert to provide comprehensive vascular access guidance. The primary components include a longitudinal linear array transducer assembly 1110 and a transverse array transducer assembly 1111 within the probe housing 2050. The longitudinal linear array transducer assembly is operable to generate real-time long-axis views of the common femoral artery and visualizes the entire needle trajectory throughout insertion while the transverse arrays are operable to provide short-axis visualization of the CFA and adjacent structures including the femoral vein, nerve, and lymphatics. In one aspect, the longitudinal array may comprise a 256-element linear array operating at 6 MHz with a 98.55 mm×6 mm aperture and the transverse phased array transducer assembly may comprise three 48-element phased arrays positioned on one side of the longitudinal array, each operating at 6 MHz with an 8.63 mm×6 mm aperture. In certain embodiments, the transducer geometry assembly may comprise exactly three transverse phased arrays strategically positioned along the longitudinal array.

The system may further comprise a depth-dependent array spacing configuration that precisely positions the transverse arrays along the probe's longitudinal axis according to expected CFA depth distribution and needle insertion angles. The system may comprise a needle-entry offset region offset relative to a patient-facing surface of the ultrasound probe enabling visualization of needle puncture for deep CFAs exceeding approximately 44 mm without requiring additional arrays. The system may comprise an acoustic boot pad 2150 that surrounds and/or overlays both array types to eliminate superficial blind spots and ensure visualization of shallow structures.

Supporting components include acoustic backing layers bonded to the piezoelectric composite rear surfaces to absorb rearward acoustic energy and prevent image-degrading reverberations; first and second acoustic matching layers providing graduated impedance transition from approximately 30 MRayl through 6 MRayl to 2.4 MRayl for optimal energy transfer; elevation focusing lenses with convex geometry and 900 m/s sound velocity focusing the beam at 40 mm depth; a 1-3 composite piezoelectric structure with optimized pillar-and-kerf geometry for 6 MHz operation and 10 μm kerfs for element isolation; flex circuit interconnects routing signals to selective-activation electronics; electromagnetic shielding layers; and visual or tactile indicators marking the desired needle entry position and offset region boundaries for clinician guidance.

The present invention relates to a handheld ultrasound probe system configured for guided percutaneous access to the common femoral artery (CFA). The system provides simultaneous orthogonal imaging planes through a specific arrangement of ultrasound transducer arrays that enables real-time visualization of both the needle trajectory and the target vessel throughout the insertion procedure.

The probe assembly comprises a housing 2050 containing multiple ultrasound transducer arrays arranged in a predetermined configuration. The housing 2050 is constructed from medical-grade polymer materials suitable for repeated disinfection and includes an ergonomic grip portion sized for single-handed operation. The patient-facing surface of the housing 2050 defines a contact plane configured to rest against the patient's skin during use.

The longitudinal linear array transducer assembly 1110 is mounted within the housing 2050 with its long axis oriented to align with the expected longitudinal axis of the CFA when the probe is properly positioned on the patient. This exemplary assembly comprises 256 piezoelectric elements arranged in a linear configuration with an array aperture measuring 98.55 mm in length and 6 mm in width. The elements are electrically connected to transmit and receive circuitry through a flexible circuit board that routes signals from each element to corresponding channels in the beamforming electronics. The 6 MHz operating frequency is selected to provide adequate penetration depth while maintaining sufficient resolution for vascular imaging applications, although other operating frequencies may be used depending on the depth and resolution needed for a given procedure as would be apparent to one of ordinary skill in the art. The extended 98.55 mm aperture length enables visualization of an extended segment of the CFA and surrounding anatomy, accommodating variations in vascular anatomy and providing sufficient field of view to track the needle throughout its insertion path.

The transverse phased array transducer assembly 1111 comprises three discrete phased array transducers positioned along one lateral side of the longitudinal linear array. Each phased array contains 48 piezoelectric elements arranged in a configuration that enables electronic beam steering and focusing. The array aperture for each phased array measures 8.63 mm by 6 mm, with the elements connected to dedicated beamforming channels that enable independent control of transmit delays and receive processing for each array. The 6 MHz operating frequency matches that of the longitudinal array to provide consistent imaging characteristics across all imaging planes, although other operating frequencies may be used depending on the depth and resolution needed for a given procedure as would be apparent to one of ordinary skill in the art.

The three transverse phased arrays are designated as proximal, middle, and distal arrays based on their position along the longitudinal axis of the probe. The spacing between these arrays follows a depth-dependent configuration that accounts for the expected range of CFA depths encountered in clinical practice. The proximal array is positioned at a first axial location, the middle array at a second axial location separated from the first by a calculated distance, and the distal array at a third axial location separated from the second by another calculated distance. These distances are determined through an optimization process that considers: (1) the statistical distribution of CFA depths in the patient population, ranging from superficial vessels at 15 mm depth to deep vessels exceeding 50 mm depth; (2) the maximum clinically acceptable needle insertion angle of 45 degrees relative to the skin surface; and (3) the electronic steering capability of the phased arrays, which provides approximately ±30 degrees of beam steering from the mechanical normal of each array.

The optimization process ensures that for any CFA depth within the expected clinical range, at least one of the three transverse arrays will capture the needle-artery intersection point within its steerable field of view. For shallow CFAs (15-25 mm depth), the proximal array provides optimal visualization. For intermediate depths (25-40 mm), the middle array captures the intersection. For deep vessels (40-50 mm), the distal array maintains visibility of the needle-artery interface. This arrangement eliminates the need for manual repositioning of the probe during the procedure, as would be required with a single transverse array design.

The needle-entry offset region (also discussed in detail below in association with FIG. 2d) is defined relative to the patient-facing surface of the probe housing as a designated area where the operator initiates needle insertion. This region is associated with a linear offset along the longitudinal direction from what would otherwise be the desired needle entry position directly adjacent to the proximal transverse array. The offset distance is calculated to accommodate visualization requirements for CFAs deeper than 44 mm, where the needle trajectory at maximum insertion angle would otherwise extend beyond the field of view of the distal transverse array. By offsetting the needle entry point, the effective imaging range is extended without requiring a fourth transverse array, thereby maintaining the compact probe design while ensuring complete procedural guidance capability across the full range of anatomical variations. Although described herein with only three transverse arrays, the system may be adapted to include more transverse arrays to image deeper regions as would be apparent to one of ordinary skill in the art. Similarly, additional transverse arrays may be used to image shallower regions wherein the focal depth ranges of each of the transverse arrays are adjusted to more narrowly cover different depth ranges as would be apparent to one of ordinary skill in the art.

The acoustic boot pad 2150, as described above, comprises a compliant acoustic coupling structure that extends from the patient-facing surface of the probe housing. The boot pad 2150 is formed from a material with acoustic impedance matched to soft tissue, such as a hydrogel or silicone-based compound with appropriate acoustic properties. The boot pad 2150 spans both the longitudinal and transverse arrays, creating a continuous acoustic coupling interface. The boot pad 2150 effectively moves the acoustic starting point away from the array elements, ensuring that superficial structures, including the skin surface and immediate subcutaneous tissues, fall within the usable imaging field of all arrays.

The probe system includes integrated electronics housed within the probe body. A multichannel beamforming system provides independent transmit and receive processing for each transducer array. The longitudinal array beamformer supports 256 channels for full synthetic aperture imaging or can operate in multiplexed mode for reduced channel count implementations. The phased array beamformers each support 48 channels with programmable delays for beam steering and dynamic focusing. Digital signal processing circuitry performs envelope detection, scan conversion, and image formation for all four imaging planes simultaneously.

A central processing unit coordinates the operation of all subsystems and implements the user interface. The processor executes algorithms for automatic optimization of imaging parameters based on detected tissue characteristics and depth measurements. Real-time image processing includes speckle reduction, edge enhancement, and automated measurement tools specific to vascular access procedures. The processor also implements safety monitoring functions, including acoustic output calculations to ensure compliance with regulatory limits for diagnostic ultrasound.

The display system 1050, as described above, comprises a high-resolution color display integrated into the probe housing or connected wirelessly or wired (e.g. via a flexible cable) to a separate display unit. The display is operable to present at least one image and may display a plurality of images simultaneously. For example, a plurality of ultrasound images may be simultaneously displayed (e.g. in a quad-view or grid-view format). In one aspect, this may comprise displaying the longitudinal view occupying a larger portion of the display area and the three transverse views arranged in a column alongside. This is merely one exemplary display option and other display variations may be used without departing from the scope of the invention as would be apparent to one of ordinary skill in the art. Color flow Doppler capability is provided for all imaging planes to distinguish arterial from venous flow and confirm vessel identification. The display includes graphical overlays indicating needle trajectory predictions, depth measurements, and anatomical labels.

Power management circuitry, which may be part of and/or controlled by adaptive electronics 1004 and/or software module 1002, enables battery-powered operation for field portability. The power system includes rechargeable lithium-ion batteries providing a minimum of two hours of continuous scanning operation. Power conditioning circuits provide stable, low-noise power rails for the sensitive analog front-end electronics. Intelligent power management reduces power consumption by selectively activating only the transducer arrays currently in use and implementing aggressive power-down modes during idle periods.

The probe includes wireless communication capabilities, e.g. embodied in data transfer interface 1001, for data transfer and remote consultation. A wireless transceiver supports standard medical device communication protocols for integration with hospital information systems. Image data can be streamed in real-time to remote displays or archived for documentation and training purposes. The wireless system includes encryption and authentication features to ensure patient data security.

Manufacturing of the probe involves precision assembly techniques to maintain the required geometric relationships between transducer arrays. The longitudinal array is fabricated using dice-and-fill methods to achieve the required element pitch and minimize crosstalk. The phased arrays utilize multilayer ceramic technology for improved bandwidth and sensitivity. All arrays are acoustically isolated from each other using absorptive backing materials to prevent acoustic crosstalk that could degrade image quality.

Calibration procedures ensure consistent performance across manufactured units. Each probe undergoes acoustic testing to verify frequency response, beam profiles, and sensitivity. Geometric calibration establishes the precise spatial relationships between the imaging planes generated by each array. This calibration data is stored in non-volatile memory within the probe and used by the image processing algorithms to ensure accurate spatial registration of the multiple views.

The user controls and/or user interface includes tactile controls positioned for single-handed operation. Dedicated buttons provide quick access to frequently used functions such as depth adjustment, gain control, and image freeze. Haptic feedback confirms button presses and mode changes without requiring visual attention to be diverted from the display.

Clinical workflow integration features include preprogrammed imaging presets optimized for CFA access procedures. The probe automatically detects when it is positioned over the femoral region based on tissue characteristics and adjusts imaging parameters accordingly. Needle enhancement algorithms use image processing to highlight the needle against surrounding tissue, improving visibility in challenging imaging conditions.

Quality assurance features include built-in test patterns and phantom imaging modes for routine performance verification. The probe maintains an internal log of usage statistics and error conditions for maintenance planning. Software updates can be installed via the wireless interface to add new features and imaging optimizations based on clinical feedback.

The modular design architecture facilitates servicing and upgrades. The transducer arrays are mounted in replaceable modules that can be exchanged without requiring complete probe disassembly. This modularity also enables future enhancements such as alternative array configurations optimized for specific clinical applications while maintaining the same basic probe platform. The present invention operates as an integrated ultrasound-guided vascular access system comprising a handheld probe with multiple transducer arrays, custom signal processing electronics, and a display interface. The probe incorporates a longitudinal array and at least three transverse arrays positioned at predetermined intervals along the probe body. These arrays work in coordination to generate simultaneous multi-planar imaging views of the target anatomical region.

The system interfaces with standard medical needles and guidewires used in vascular access procedures. The probe's physical design includes a needle guide or reference point on its surface that establishes a consistent spatial relationship between the probe position and the needle insertion site. This relationship enables the system to predict and display the needle trajectory relative to the imaged anatomy. The custom electronics receive and process ultrasound signals from all arrays simultaneously, applying beamforming and image reconstruction algorithms to generate composite images that display both longitudinal and transverse views on a single screen or display device.

The invention operates within the broader medical environment by addressing limitations of conventional single-plane ultrasound systems. The multi-array configuration provides depth-specific imaging planes that correspond to clinically relevant vessel depth ranges. This design allows clinicians to select the appropriate transverse imaging plane based on the measured vessel depth, thereby maintaining optimal visualization throughout the needle insertion procedure. The system's battery-powered operation enables deployment across various clinical settings without dependency on facility infrastructure. The portable nature of the device facilitates its use in emergency medical services, bedside procedures, and resource-limited environments where traditional ultrasound equipment may be impractical or unavailable.

The system interacts with the patient's anatomy through acoustic coupling between the probe surface and the skin, typically facilitated by ultrasound gel. The longitudinal array generally provides continuous visualization along the vessel axis (depending on vessel geometry), while the transverse arrays capture cross-sectional anatomy at specific depths. This multi-planar imaging approach enables simultaneous monitoring of critical anatomical relationships, including the spatial orientation of the common femoral artery, femoral vein, and femoral nerve. Moreover, this multiplanar imaging approach may allow for 3D visualization of the target vessel and surrounding anatomy by combining the longitudinal and transverse images at various locations to generate a 3D volumetric rendering. The real-time image processing and display allow the clinician to make immediate adjustments to needle trajectory based on visual feedback, thereby reducing the risk of complications associated with blind or single-plane guided vascular access procedures.

The longitudinal linear array transducer assembly 1110 may comprise a 1-3 composite piezoelectric structure utilizing PZT-5H material with a piezoelectric layer having a thickness of 240 μm. The assembly 1110 includes array elements configured at a pitch of 0.385 mm, corresponding to 1.52 at 6 MHz operating frequency, with individual element dimensions of 0.375 mm×6 mm. Adjacent elements are separated by 10 μm kerfs. The 1.52 pitch configuration enables suppression of grating lobe artifacts while maintaining a reduced total element count, providing needle visibility at steering angles up to 45° with a ±10° acceptance range.

The composite structure of assembly 1110 incorporates a 40% volume fraction configuration with 52 μm pillars and 30 μm kerfs filled with Epotek 301 epoxy. During operation, the assembly 1110 employs a sliding sub-aperture technique wherein 48 elements are sequentially activated to form transmit and receive beams. This selective activation pattern maintains image quality in regions where needle visualization is anticipated while reducing instantaneous power consumption. The assembly 1110 provides lateral resolution ranging from 0.34 mm at 20 mm depth to 1.17 mm at 70 mm depth with 0.4 mm line spacing.

The longitudinal linear array assembly 1110 further comprises an acoustic backing layer consisting of alumina-loaded Epotek 301 epoxy with 30% volume fraction alumina and 11 mm thickness for absorbing rearward-propagating acoustic energy. The assembly 1110 includes two acoustic matching layers: a first matching layer of alumina-loaded Epotek 301 having 30% volume fraction, 120 μm thickness, and approximately 6 MRayl impedance; and a second water-clear matching layer having 95 μm thickness and approximately 2.4 MRayl impedance. These matching layers optimize acoustic energy transfer between the piezoelectric elements and the imaging medium.

A convex lens element of the assembly 1110 comprises alumina-loaded silicone containing 44% alumina by weight, exhibiting 1.3-1.4 MRayl impedance and 900 m/s sound speed. The lens provides elevation focusing at 40 mm depth with a 28.4 mm radius of curvature and 160 μm maximum thickness. The convex-convex lens geometry facilitates acoustic coupling to an acoustic boot pad interface.

The assembly 1110 interfaces with selective-activation electronics that drive a subset of elements at any given moment, enabling power-efficient operation. The assembly 1110 also interfaces with an acoustic boot pad that provides acoustic coupling to the patient surface during imaging procedures.

Alternative embodiments of the longitudinal linear array transducer assembly 1110 may incorporate single-crystal piezoelectric materials such as PMN-PT in place of the PZT-5H composite, potentially providing enhanced sensitivity and bandwidth characteristics, though with increased material costs. Other implementations may utilize standard 1.02 pitch arrays with higher element counts (384 or more elements) to achieve comparable aperture dimensions without grating lobe considerations, though such configurations would increase channel count and power consumption beyond levels suitable for portable operation.

Capacitive Micromachined Ultrasonic Transducer (CMUT) technology represents another alternative implementation for the assembly 1110, potentially offering improved manufacturing consistency and enhanced integration capabilities with electronic components. A reduced array aperture configuration (for example, 64 mm) combined with mechanical translation mechanisms could be employed, though such implementations would not provide the real-time extended field-of-view capability for needle trajectory visualization offered by the described embodiment of assembly 1110.

The transverse phased array transducer assembly 1111 comprises three independent phased array transducers positioned at predetermined axial locations along the device housing. Each phased array within assembly 1111 incorporates 48 piezoelectric elements arranged in a linear configuration with an element pitch of 0.180 mm, corresponding to 0.7 wavelengths at the 6 MHz operating frequency. Individual elements measure 0.170 mm in width by 6 mm in elevation, with 10 μm kerf separation between adjacent elements.

The 0.72 pitch configuration of assembly 1111 provides beam steering capability while suppressing grating lobe formation during sector scanning operations. This pitch dimension enables electronic beam deflection across the required angular range without mechanical translation of the transducer elements. Assembly 1111 operates in a line-by-line imaging mode, employing dual transmit focal zones positioned at 25 mm and 50 mm depth to optimize image resolution throughout the 5 mm to 70 mm imaging range.

Each phased array within assembly 1111 may utilize a multilayer acoustic stack construction. The active layer comprises 1-3 composite PZT-5H piezoelectric material having a thickness of 240 μm and 40% ceramic volume fraction. An 11 mm thick backing layer of alumina-loaded Epotek 301 (30% alumina content by volume) provides acoustic damping. Two quarter-wavelength matching layers, measuring 120 μm and 95 μm respectively, facilitate acoustic impedance matching between the piezoelectric elements and tissue. An alumina-loaded silicone lens provides elevation focusing at 40 mm.

The three phased arrays of assembly 1111 are positioned at 8.7 mm, 17.9 mm, and 36.9 mm from the distal end of the longitudinal array, respectively. These positions correspond to calculated optimal locations based on common femoral artery depth distributions and maximum acceptable needle insertion angles. The positioning ensures needle visibility remains within the electronic steering limits of each phased array during vascular access procedures. While the transverse arrays are disclosed as being positioned at particular locations/distances along the length of the longitudinal array, these locations are exemplary and could be adjusted, as would be apparent to one of ordinary skill in the art, as a function of target depth and/or depth distributions for a given application. This depth dependent spacing is discussed in more detail below.

Assembly 1111 achieves lateral resolution ranging from 0.72 mm at 20 mm depth to 2.51 mm at 70 mm depth, enabling visualization of vessels having diameters as small as 5 mm throughout the specified depth range. The phased array configuration permits electronic sector scanning without mechanical movement, providing real-time imaging capability during needle guidance procedures.

During operation, assembly 1111 interfaces with selective-activation electronics that energize fewer than all three arrays simultaneously. Each active array utilizes a subset of available elements during beam steering operations to reduce overall power consumption. This selective activation scheme enables extended battery operation while maintaining imaging performance.

Alternative embodiments of assembly 1111 may incorporate four or more transverse phased arrays to provide increased depth resolution. Such configurations would require additional electronic channels, more signal multiplexers, and/or increased power consumption. Alternative element counts of 64 or 96 elements per array would provide improved lateral resolution but would require proportionally more system channels and power resources.

Curved linear array configurations could substitute for the phased arrays of assembly 1111, eliminating beam steering requirements but sacrificing the sector scanning capability required for complete angular coverage at the specified depth range. Matrix array configurations could theoretically enable arbitrary imaging plane selection but would substantially increase element count, interconnect complexity, and computational requirements beyond practical limits for handheld implementation.

The transverse phased array transducer assembly 1111 may alternatively employ different piezoelectric materials such as PMN-PT single crystals or lead-free ceramics, provided they maintain comparable electromechanical coupling and acoustic impedance characteristics. Alternative backing materials including tungsten-loaded epoxy or porous ceramics may substitute for the alumina-loaded backing, with appropriate adjustments to thickness and acoustic impedance. The matching layer materials and thicknesses may be modified based on the selected piezoelectric material and target tissue impedance to maintain efficient acoustic energy transfer.

The depth-dependent array spacing configuration 1120 comprises a predetermined arrangement of three transverse phased arrays positioned at calculated intervals along the longitudinal axis of the probe housing. The configuration 1120 establishes array positions at defined locations measured from the longitudinal array end, creating three distinct but overlapping depth coverage zones for needle visualization during common femoral artery (CFA) cannulation procedures. In one embodiment, the defined locations comprise 8.7 mm, 17.9 mm, and 36.9 mm from the longitudinal array end, although these could be adjusted without departing from the scope of the invention as would be apparent to one of ordinary skill in the art.

The configuration 1120 operates by exploiting the geometric relationship between array position, needle insertion angle, and visualization depth. When a needle is introduced from the probe face toward a CFA target at the desired 45° insertion angle, the needle trajectory intersects different transverse array imaging regions depending on the vessel depth. Array 1, positioned at 8.7 mm, generates an imaging region that captures needle punctures for CFA depths ranging from 5 mm to 10.3 mm. Array 2, positioned at 17.9 mm, provides coverage from 10.3 mm to 21.3 mm. Array 3, positioned at 36.9 mm, extends coverage from 21.3 mm to 44 mm. The overlapping boundaries at 10.3 mm and 21.3 mm ensure continuous visualization capability across the entire depth range. It is noted that these dimensions are exemplary and may be adjusted depending on other features of the transducer, such as, but not limited to, operating frequency, number of elements, etc., as would be understood by a person of ordinary skill in the art.

The spacing values of configuration 1120 are derived through geometric calculations that account for multiple constraints. The 45° desired insertion angle establishes the primary trajectory geometry. The ±10° angular acceptance range, determined by the transverse array pitch and grating lobe characteristics, defines the effective steering limits within which reliable imaging and needle conspicuity are maintained. The configuration 1120 incorporates statistical distribution data of CFA depths in the target patient population, which extends to a maximum imaging depth of 70 mm. These parameters combine to yield the specific spacing values that optimize needle visualization while maintaining image quality within acceptable steering angle limits for each phased array.

The configuration 1120 interfaces with the needle-entry offset region to extend effective depth coverage beyond the 44 mm limit of Array 3. This interaction enables visualization of deeper vessels without requiring additional transverse arrays or exceeding the probe's power and channel count constraints. The fixed nature of configuration 1120 within the probe housing eliminates mechanical complexity and potential failure modes associated with adjustable positioning systems.

Alternative implementations to configuration 1120 include uniform spacing arrangements where transverse arrays are positioned at equal intervals along the probe length. Such configurations would simplify manufacturing but would fail to account for the non-linear relationship between array position and visualized depth when needles are inserted at oblique angles. The uniform spacing would result in either gaps in depth coverage or excessive overlap, reducing efficiency.

Dynamic positioning systems represent another alternative approach, where motorized mechanisms adjust array positions based on real-time vessel depth measurements. While such systems could theoretically optimize array placement for each patient, they introduce mechanical complexity, increased weight, additional power consumption, and potential failure modes that compromise the reliability requirements for handheld medical devices. The added components would also increase probe dimensions beyond ergonomic limits for single-handed operation.

Higher density array configurations with more than three transverse arrays at closer spacing intervals could provide finer depth resolution and smaller coverage gaps. However, such configurations could exceed the power budget and channel count limitations imposed by portable ultrasound system architectures. Each additional array may require dedicated transmit/receive channels, additional signal multiplexers, beamforming resources, and/or power allocation which may come with drawbacks such as larger, heavier system components incompatible with point-of-care deployment requirements, increased signal loss, parasitic capacitance and crosstalk, increased circuitry complexity, and increased cost.

The configuration 1120 may also be implemented with variable array sizes, where arrays positioned for deeper imaging employ larger apertures to compensate for increased attenuation. This variation would maintain consistent image quality across the depth range while adhering to the same spacing principles. Alternatively, the configuration 1120 could incorporate arrays with different operating frequencies, with higher frequencies for shallow imaging and lower frequencies for deeper penetration, while maintaining the same physical spacing relationships.

FIG. 2d depicts an exemplary needle entry offset region according to an embodiment of the invention. The needle-entry offset region 400 comprises a designated area relative to the probe surface positioned at a distance from the probe along the longitudinal axis relative to the desired needle entry position. This region 400 provides an alternative needle insertion location that modifies the geometric relationship between the needle trajectory and the ultrasound arrays when imaging vessels at depths exceeding 44 mm. The probe 1000 may comprise a light projecting component 2100 (e.g. a laser, LED, etc.) operable to project light onto the skin surface at the approximate offset location 400 where the needle 450 should be inserted thereby illuminating the skin to provide a visual indication to a user of a desired location to insert the needle that leverages this needle offset region in order to reach deeper depths while still being able to image the anatomy and needle advancement with the disclosed probe and transducer configuration. In an alternate configuration, the probe may comprise a camera, in place of light projecting component, for example, or mounted on an attached needle guide assembly, wherein the camera is directed at the general offset region. Via image processing and overlay techniques, the display of the handheld ultrasound device may display an image of the skin surface captured by the camera along with the offset region marked via graphical indicators or overlays thereby providing real-time guidance for the user in inserting the needle at a preferred offset distance from the probe.

The offset region 400 operates by increasing the horizontal distance between the needle entry point and the target vessel location. For imaging and needle guidance towards vessels at depths less than approximately 44 mm, the needle will generally be inserted at a desired location adjacent to the probe housing with the needle potentially touching the probe housing (which may include a groove) thereby using the probe housing (or an attached needle guide) as guide for the needle. When a clinician identifies a common femoral artery (CFA) at depths greater than 44 mm using the imaging arrays, the needle insertion point is shifted from the desired position to the offset region 400. This displacement along the longitudinal axis creates additional horizontal travel distance for the needle before reaching the target depth. The increased horizontal component enables the needle to maintain an insertion angle at or below the predetermined preferred maximum of 45° (±10°) while still intersecting the imaging field of Array 3 at the desired puncture depth.

The offset region 400 may be identified via visual indicators to facilitate proper needle positioning. These indicators may include visual projections that guide the clinician to the appropriate insertion location based on the identified vessel depth. The offset distance is geometrically determined by the relationship between Array 3's position at 36.9 mm from the probe tip, the array's maximum visualization depth of 44 mm when using desired entry position, the target maximum imaging depth of 70 mm, and the acceptable insertion angle range.

The offset region 400 functions in conjunction with the depth-dependent array spacing configuration to provide comprehensive depth coverage. While the array spacing handles vessels at shallow to moderate depths through angle adjustment, the offset region 400 addresses deep vessel access through positional adjustment. This complementary approach enables full clinical depth coverage without requiring additional transverse arrays.

Alternative implementations to the offset region 400 include incorporating a fourth transverse phased array positioned further along the longitudinal axis. This approach would provide direct imaging coverage for deeper puncture depths but would require additional transducer elements (e.g. another 48 element transverse phased array) with corresponding increases in channel count, power consumption, and system cost. The probe dimensions would also increase to accommodate the additional array.

Another alternative involves implementing motorized translation or articulation mechanisms to dynamically adjust array positions relative to the needle entry point. This approach would allow real-time optimization of the geometric relationship between arrays and needle trajectory but would introduce mechanical complexity, potential reliability concerns, and increased manufacturing costs.

Software-based solutions could employ advanced needle trajectory prediction algorithms without physical offset guidance. However, such approaches would not address the fundamental geometric constraints imposed by phased array beam steering limitations and would rely on computational predictions rather than direct visualization of the needle-tissue interaction.

The offset region 400 may also be implemented with multiple discrete offset positions rather than a single offset location, providing graduated adjustment options for different depth ranges. Variable offset implementations could include sliding or rotating mechanisms that allow continuous adjustment of the entry position, though these would add mechanical complexity compared to the fixed offset design.

The acoustic boot pad 2150 comprises a specialized acoustic coupling structure positioned between the ultrasound transducer arrays and the patient contact surface. The acoustic boot pad 2150 consists of a polymeric or elastomeric material having predetermined acoustic properties, including an acoustic impedance in the range of 1.3 to 1.7 MRayl and a sound velocity between 1400 and 1600 m/s, selected to approximate the acoustic properties of human soft tissue.

The acoustic boot pad 2150 functions as an acoustic standoff element that extends the acoustic propagation path between the transducer elements and the target imaging region. In operation, ultrasound waves generated by the transducer arrays propagate through the acoustic boot pad 2150 before entering the patient's tissue. This additional propagation distance shifts the effective near-field region of the transducer arrays away from the skin surface, thereby enabling imaging of superficial anatomical structures that would otherwise fall within the near-field blind spot of the transverse phased array configuration.

The acoustic boot pad 2150 exhibits a thickness dimension selected based on the operating parameters of the transverse phased array, specifically accounting for the 6 MHz center frequency, 48-element count, and 0.7λ inter-element pitch configuration. The thickness is determined such that the near-field blind spot, which results from the constructive and destructive interference patterns inherent in phased array beam formation, is displaced to a position above the minimum expected depth of the common femoral artery (CFA), which may be as shallow as 5 mm below the skin surface.

The acoustic boot pad 2150 interfaces with the convex-convex lens geometry of both the longitudinal and transverse arrays through conformal contact surfaces. The pad material exhibits low acoustic attenuation characteristics, typically less than 0.5 dB/cm/MHz, to minimize signal loss during transit through the standoff medium. The acoustic impedance of the boot pad 2150 is selected to minimize reflection coefficients at both the transducer-pad interface and the pad-tissue interface, thereby maximizing acoustic energy transmission into the patient.

Alternative implementations of the acoustic standoff function include disposable gel pads that may be applied between the transducer and patient. Such gel pads provide variable standoff distances but may introduce positioning uncertainties and require replacement between procedures. Fluid-filled coupling systems, such as water bags or saline-filled membranes, offer adjustable standoff distances but introduce mechanical complexity and potential failure modes associated with fluid containment.

Another alternative involves the use of solid delay lines fabricated from materials such as polymethylpentene (TPX) or cross-linked polystyrene, which provide stable acoustic properties and precise dimensional control. These materials may be machined or molded to specific geometries but may exhibit higher acoustic impedance mismatches compared to tissue-mimicking elastomers.

Electronic beam steering modifications could partially compensate for near-field limitations by adjusting the phased array timing sequences, though such approaches cannot fully recover imaging capability in regions where acoustic coupling is geometrically prevented. Synthetic aperture techniques might extend the effective aperture of the transverse array but would require modifications to the acquisition sequence and signal processing architecture.

The acoustic boot pad 2150 may incorporate acoustic scattering particles or microbubbles to provide reference echoes for system calibration or to enhance visualization of the standoff layer boundaries during imaging. The pad material may further include antimicrobial additives or surface treatments to facilitate cleaning and disinfection between patient uses.

The ultrasound probe may comprise additional elements to support operation as described herein. The ultrasound probe may comprise acoustic backing layers comprising alumina-loaded Epotek 301 epoxy with a 30% volume fraction and 11 mm thickness, bonded to the rear surface of the piezoelectric composite in both the longitudinal and transverse array assemblies. These backing layers absorb rearward-propagating acoustic energy to reduce reverberations that could degrade image quality. The first acoustic matching layers consist of alumina-loaded Epotek 301 with 120 μm thickness and approximately 6 MRayl impedance, while the second acoustic matching layers comprise water-clear material with 95 μm thickness and approximately 2.4 MRayl impedance. These matching layers are sequentially bonded to the front surface of the piezoelectric composite, providing graduated acoustic impedance transition from the high-impedance piezoelectric material (~30 MRayl) through intermediate impedance to near-tissue impedance, thereby enhancing acoustic energy transfer into the patient.

The elevation focusing lenses comprise alumina-loaded silicone with 44% alumina by weight, featuring convex geometry with 40 mm focal depth, 28.4 mm radius of curvature, and 160 μm maximum thickness. These lenses are bonded atop the matching layers and utilize their convex geometry and controlled sound velocity (900 m/s) to focus the acoustic beam in the elevation dimension at 40 mm depth for optimizing slice thickness during CFA visualization. The 1-3 composite piezoelectric elements consist of PZT-5H material with 240 μm thickness, 40% volume fraction, 52 μm pillars, and 30 μm kerfs filled with Epotek 301, providing electromechanical transduction with pillar-and-kerf geometry optimized for bandwidth and sensitivity at 6 MHz. Element kerfs of 10 μm width separate array elements to provide acoustic and electrical isolation. Flex circuit interconnects route electrical signals from the array elements to the selective-activation electronics module, while shielding layers prevent electromagnetic interference. The cable assembly or wireless communication module enables data transmission, and visual/tactile needle entry position indicators on the probe housing surface mark the desired needle entry position and offset region boundaries to guide clinician positioning.

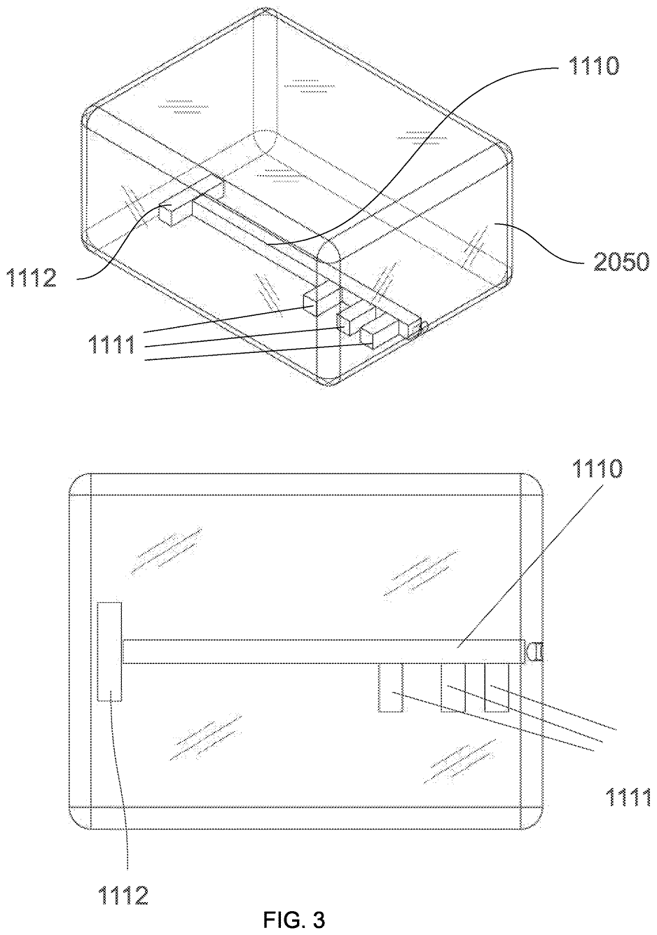

FIG. 3 illustrates an exemplary configuration of a transducer assembly according to an exemplary embodiment of the invention. In this embodiment, the transducer configuration comprises a fourth transverse array 1112 positioned at an opposite end of the longitudinal array relative to the location of the three transverse phased arrays 1111. In use, this fourth array would be positioned most proximal to the heart. Unlike the other three transverse arrays, the fourth array does not partake in the process of ultrasound imaging of arterial cannulation, and thus does not have the same positional or spacing requirements. The fourth transverse array 1112 generally serves to image the underlying anatomy for purposes of scouting the topology of the patient physiology and determining critical anatomical landmarks. This topology would be advantageous when trying to map out the patient vasculature (e.g. CFA) and corresponding anatomical landmarks. For instance, if the user were to begin scanning the patient in a distal to proximal direction (from the feet towards the head of the patient), as the probe approaches anatomy of interest (e.g. the CFA bifurcation), this fourth transverse array 1112 is operable to provide images that aid in determining the location of the landmarks of interest. For example, in the context of locating the CFA, this fourth transverse array 1112 may be used to identify the CFA bifurcation first (e.g. using Doppler and image recognition), and then subsequently provide guidance to the user on how to center the longitudinal array of device over the patient's CFA. By associating position and rotation information to each stored ultrasound image acquired by the fourth transverse array 1112 (optionally in combination with images from the longitudinal array 1110 and/or transverse arrays 1111), the device is operable to generate an ultrasound image library of the CFA in one sweeping motion of the ultrasound probe over the area of interest. As the ultrasound probe is moved, the device may provide feedback to the user informing the user when to rotate and how to position the device. This ultrasound image library, with corresponding image position and/or rotation information, allows the system to generate a 3D volumetric image of the CFA and surrounding structures. This fourth array 1112 and subsequent image processing serves to help a user navigate the probe to a desired location and can be used to determine whether the target anatomy (e.g. CFA) is at a depth suitable for imaging with one or more of the three transverse arrays 1111 and/or whether the needle offset region 400 should be used for imaging the needle during needle insertion and advancement.

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments).

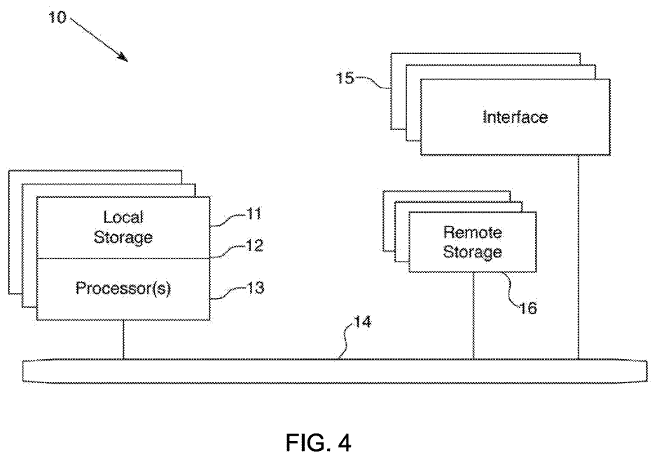

Any of the above mentioned systems, units, modules, engines, controllers, interfaces, components or the like may be and/or comprise hardware and/or software as described herein. For example, the processing system 103, the network 150, and subcomponents thereof may be and/or comprise computing hardware and/or software as described herein in association with FIGS. 4-7. Furthermore, any of the above mentioned systems, units, modules, engines, controllers, interfaces, components or the like may use and/or comprise an application programming interface (API) for communicating with other systems units, modules, engines, controllers, interfaces, components, or the like for obtaining and/or providing data or information.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software-or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

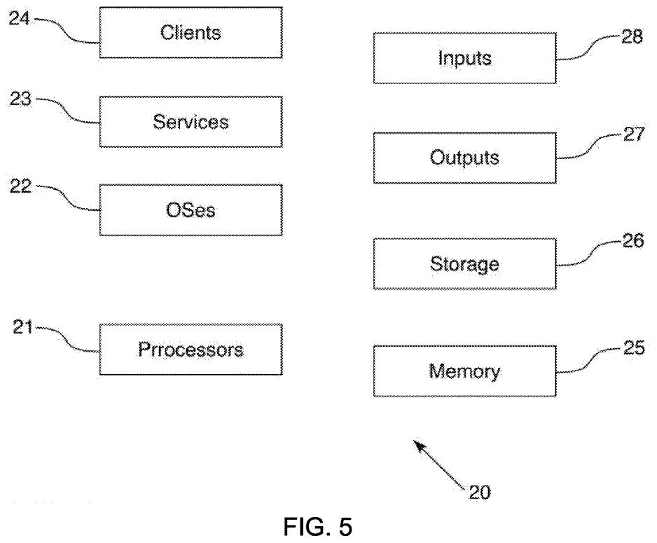

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

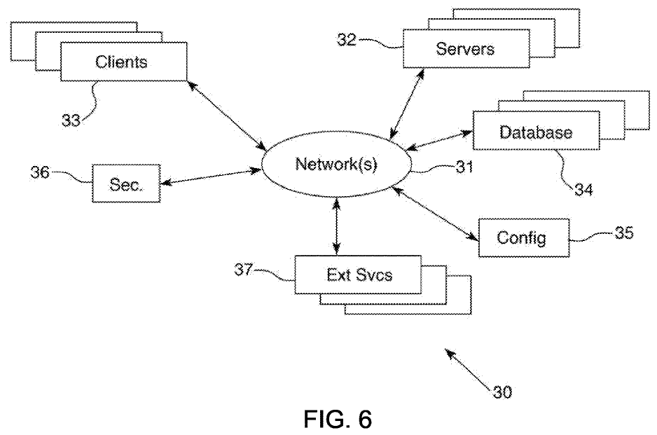

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

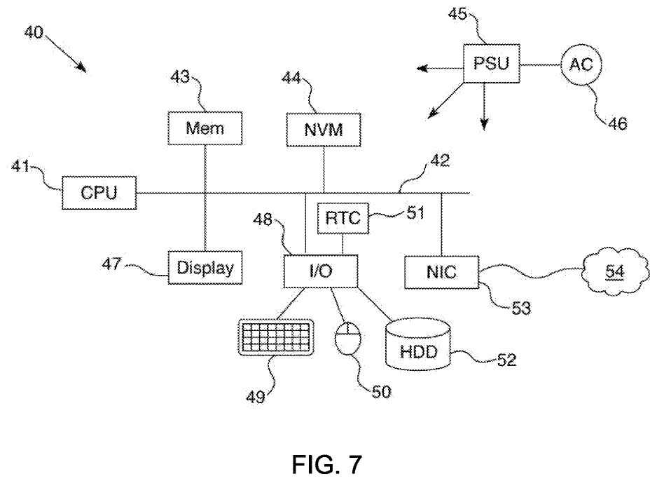

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. An ultrasound probe for ultrasound-guided cannulation of a target blood vessel, comprising:
a probe body having a longitudinal axis extending along a patient-facing surface of the probe body;
a longitudinal linear transducer array disposed in the probe body and extending along the longitudinal axis, the longitudinal linear transducer array being configured to generate real-time longitudinal ultrasound images in a longitudinal plane;
a plurality of transverse phased arrays disposed in the probe body on along the longitudinal linear transducer array, each transverse phased array having a respective transverse imaging plane oriented substantially perpendicular to the longitudinal axis and being configured to generate real-time transverse ultrasound images in the transverse imaging plane;
wherein the plurality of transverse phased array comprises at least three transverse phased arrays, each positioned at a different location along the longitudinal axis;
wherein an axial spacing between the transverse phased arrays is selected such that, for a set of different depths of a target blood vessel within a predefined access region, a needle introduced toward the target blood vessel can be imaged within a predetermined maximum insertion angle relative to the longitudinal axis while remaining within a steering range of at least one of the transverse phased arrays;
an acoustic pad comprising an acoustic coupling material arranged as a boot on the probe body, the acoustic pad having a thickness selected to reduce a superficial blind spot for the transverse phased arrays when imaging tissue superficial to the target blood vessel; and
adaptive probe electronics disposed within the probe body and electrically coupled to the longitudinal linear transducer array and to the transverse phased array, the probe electronics configured to adaptively adjust operation of the transducer arrays based on target depth and/or inserted needle progression.

2. The ultrasound probe of claim 1, wherein the longitudinal linear transducer array has an element pitch in a range of about 1.2 to about 1.8 acoustic wavelengths at an operating frequency between about 5 MHz and about 8 MHz.

3. The ultrasound probe of claim 1, wherein each of the transverse phased arrays comprises transducer elements having an element pitch in a range of about 0.5 to about 0.9 acoustic wavelengths at the operating frequency.

4. The ultrasound probe of claim 1, wherein the transverse phased arrays are configured to image at least the common femoral artery, a femoral vein, and a femoral nerve in each transverse imaging plane.

5. The ultrasound probe of claim 1, wherein the predetermined maximum insertion angle is between about 20 degrees and about 45 degrees relative to the patient-facing surface.

6. The ultrasound probe of claim 1, wherein the acoustic pad has a uniform thickness profile that is equal across the transverse phased arrays and the longitudinal linear transducer array.

7. The ultrasound probe of claim 1, wherein the acoustic pad is formed as a removable boot configured to be mechanically attached to and detached from the probe body.

8. The ultrasound probe of claim 1, wherein the probe electronics are configured to dynamically select which one of the transverse phased arrays is active based on a detected depth of the target blood vessel.

9. The ultrasound probe of claim 1, further comprising a wireless or wired connector configured to couple the probe electronics to an internal or external imaging system that generates images including at least one longitudinal image from the longitudinal linear transducer array and at least one transverse image from one of the transverse phased arrays.

10. The ultrasound probe of claim 1, wherein the probe body, the longitudinal linear transducer array, the transverse phased arrays, the probe electronics, and a power source are contained within a handheld housing.

11. The ultrasound probe of claim 1, wherein the target blood vessel comprises the common femoral artery.

12. The ultrasound probe of claim 1, wherein the longitudinal images comprise at least a target blood vessel and an insertion trajectory of an inserted needle and the transverse images comprise at least the target blood vessel and surrounding anatomical structures.

13. The ultrasound probe of claim 1, wherein the transverse phased arrays are disposed in the probe body on only one side the longitudinal linear transducer array.

14. The ultrasound probe of claim 1, wherein the adaptive probe electronics are operable to:
control the longitudinal linear transducer array to operate with a subset of its transducer elements active at a given time to generate the longitudinal images; and
control the transverse phased arrays to operate fewer than all of the transverse phased arrays concurrently and to limit a number of active elements per transverse phased array transducer during beam steering,
such that an instantaneous power consumption and channel count of the probe electronics are reduced to enable battery-powered handheld operation of the ultrasound probe while providing simultaneous longitudinal and transverse visualization of the target blood vessel and the needle.

15. A method for ultrasound-guided cannulation of a target blood vessel of a patient using the ultrasound probe of claim 1, comprising:
positioning the ultrasound probe on a region of the patient such that at least one of the transverse phased arrays images the target blood vessel in a transverse imaging plane;
determining a depth of the target blood vessel relative to the patient-facing surface;
selecting, based on the depth, a needle-entry position (and angle) aligned with a central one of the transverse phased arrays;
inserting a needle toward the target blood vessel from the selected position at an insertion angle that does not exceed the predetermined maximum insertion angle; and
concurrently displaying:
a longitudinal image generated from the longitudinal linear transducer array, and
a transverse image generated from one of the transverse phased arrays,
such that the target blood vessel and the needle are visualized in both the longitudinal and transverse images during cannulation.

16. The method of claim 15, further comprising dynamically switching which one of the transverse phased arrays is active during the procedure in response to changes in at least one of the detected depth and a detected position of the target blood vessel.

17. The method of claim 15, wherein inserting a needle comprises inserting the needle at a needle-entry region defined relative to the probe body and linearly offset along the longitudinal axis from a needle-entry position aligned with a central one of the transverse phased arrays, the linear offset being configured such that, for target blood vessels deeper than a threshold depth, an insertion of the needle from the needle-entry region toward the target blood vessel occurs at an insertion angle that remains within the predetermined maximum insertion angle while the needle is visualized in at least one of the longitudinal plane and the transverse imaging planes, whereby the linear offset obviates a need for a fourth transverse phased array transducer.

18. The method of claim 17, wherein the linear offset between the needle-entry region and the needle-entry position is in a range selected based on the threshold depth so that a needle trajectory from the needle-entry region intersects the target blood vessel within the predetermined maximum insertion angle for the threshold depth.
